(19) United States
(12) Patent Application Publication
(10) Pub. No.: US 2009/0018371 A1
(43) Pub. Date: Jan. 15, 2009

(54) METHODS AND APPARATUS FOR PRODUCING ALCOHOLS FROM SYNGAS

(75) Inventors: Robert E. KLEPPER, Arvada, CO (US); Arie GEERTSEMA, Westminster, CO (US); Heinz Juergen ROBOTA, Arvada, CO (US); Ronald C. STITES, Brighton, CO (US); Richard RIDLEY, Loveland, CO (US)

Correspondence Address: Range Fuels, Inc.;Attn: Ryan O'Connor, 11101 West 120th Ave. Suite 200, Broomfield, CO, 80021, US

(73) Assignee: Range Fuels, Inc., Broomfield, CO (US)

(21) Appl. No.: 12/166,203

(22) Filed: Jul. 1, 2008

Related U.S. Application Data
(60) Provisional application No. 60/948,650, filed on Jul. 9, 2007.

(51) Int. Cl. C07C 29/32

(52) U.S. Cl. 568/902.2

(57) ABSTRACT

Methods and apparatus for producing alcohols from syngas are disclosed herein. In some variations, syngas is catalytically converted to methanol in a first reaction zone, and residual syngas from the first reaction zone is then catalytically converted to ethanol in a second reaction zone. Also, in some variations, syngas is catalytically converted to methanol in high yield in a first reaction zone, and the methanol is then converted (optionally, with additional syngas) to ethanol in a second reaction zone.

### PRIORITY DATA

This patent application claims priority under 35 U.S.C. §120 from U.S. Provisional Patent Application No. 60/948,650 for “Methods and Apparatus for Producing Alcohols from Syngas” which is hereby incorporated by reference herein for all purposes.

### FIELD OF THE INVENTION

The present invention generally relates to processes for the conversion of carbonaceous feedstocks, such as cellulosic biomass, into synthesis gas, and to processes for the conversion of synthesis gas to products such as alcohols (e.g., ethanol).

### BACKGROUND OF THE INVENTION

Ethanol and alcohol mixtures including ethanol may be used as fuels and fuel additives in place of petroleum-based products such as gasoline. Such use of alcohols can reduce the need to import petroleum. In addition, the substitution of alcohols for petroleum-based fuels and fuel additives can be particularly environmentally friendly when the alcohols are produced from feedstocks other than fossil fuels.

One synthetic route to alcohols is through catalytic processes for the conversion of syngas to alcohols. Syngas (or synthesis gas) is a mixture of monoxide (CO) and hydrogen (H₂). Generally, syngas may be produced from any carbonaceous material. In particular, biomass such as, for example, agricultural wastes, forest products, grasses, and other cellulosic material may be converted to syngas.

There exist a variety of conversion technologies to turn these feedstocks into syngas. Conversion approaches can utilize a combination of one or more steps comprising gasification, pyrolysis, steam reforming, and/or partial oxidation of a carbonaceous material.

Since the 1920s it has been known that mixtures of methanol and other alcohols can be obtained by reacting syngas over certain catalysts (Forzatti et al., Cat. Rev.—Sci. and Eng. 33(1-2), 109-168, 1991). Fischer and Tropsch observed around the same time that hydrocarbon-synthesis catalysts produced linear alcohols as byproducts (Fischer and Tropsch, Brennst.—Chem. 7:97, 1926).

However, improved methods and apparatus to convert syngas into alcohols, such as ethanol, are currently needed.

### SUMMARY OF THE INVENTION

In some embodiments, the present invention provides a method of producing one or more C₂-C₄ alcohols, the method comprising:
(i) introducing syngas into a first reaction zone comprising at least a first catalyst;
(ii) converting a portion of the syngas to methanol with the first catalyst;
(iii) introducing syngas and methanol from the first reaction zone into a second reaction zone comprising at least a second catalyst; and
(iv) converting at least a portion of the syngas and methanol introduced into the second reaction zone with the second catalyst to produce a product stream comprising one or more C₂-C₄ alcohols, such as ethanol, 1-propanol, or 1-butanol.

The second reaction zone can be in the same reactor, or in a different reactor, than the first reaction zone.

In some embodiments, the syngas introduced into the first reaction zone has an initial H₂/CO ratio, conversion of syngas to methanol in the first reaction zone causes the syngas introduced into the second reaction zone to have a second H₂/CO ratio, and the second H₂/CO ratio provides an increased yield to one or more C₂-C₄ alcohols in the second reaction zone compared to that which would be provided by the initial H₂/CO ratio. The second H₂/CO ratio is preferably lower than the initial H₂/CO ratio.

In some embodiments, the method further comprises introducing additional methanol into the second reaction zone and converting at least a portion of the additional methanol introduced into the second reaction zone to one or more C₂-C₄ alcohols with the second catalyst. In certain embodiments, at least a portion of the additional methanol introduced into the second reaction zone was previously recovered from the product stream. In some methods, additional syngas (which is not unreacted syngas from the first reaction zone) is introduced into the second reaction zone, followed by converting at least a portion of the additional syngas introduced into the second reaction zone to one or more C₂-C₄ alcohols with the second catalyst. Syngas can be recovered from the product stream and recycled through at least one of the reaction zones.

The first catalyst can comprise a material selected from the group consisting of ZnO/Cr₂O₃, Cu/ZnO, Cu/ZnO/Al₂O₃, Cu/ZnO/Cr₂O₃, Cu/ThO₂, Co/S, Mo/S, Co/Mo/S, Ni/S, Ni/Mo/S, Ni/Co/Mo/S, and any of the foregoing in combination with Mn and/or V. The first catalyst preferably includes a basic promoter.

The second catalyst can comprise a material selected from the group consisting of ZnO/Cr₂O₃, Cu/ZnO, Cu/ZnO/Al₂O₃, CuO/CoO, CuO/CoO/Al₂O₃, Co/S, Mo/S, Co/Mo/S, Rh/Ti/SiO₂, Rh/Mn/SiO₂, Rh/Ti/Fe/Ir/SiO₂, Rh/Mn/MCM-41, Ni/S, Ni/Mo/S, Ni/Co/Mo/S, and any of the foregoing in combination with Mn and/or V. The second catalyst preferably includes a basic promoter. The first catalyst and the second catalyst can, in some embodiments, have substantially the same initial composition.

In some embodiments, the invention provides a method of producing one or more C₂-C₄ alcohols, the method comprising:
(i) introducing a first amount of syngas into a first reaction zone comprising at least a first catalyst;
(ii) converting at least a portion of the first amount of syngas to methanol with the first catalyst;
(iii) introducing the methanol to a second reaction zone comprising at least a second catalyst;
(iv) introducing a second amount of syngas to the second reaction zone; and
(v) reacting at least a portion of the methanol introduced into the second reaction zone with at least a portion of the second amount of syngas with the second catalyst to produce a product stream comprising one or more C₂-C₄ alcohols.

The second amount of syngas can include syngas that did not react in the first reaction zone. The second amount of syngas additionally can include syngas that was separated and recycled from the product stream. Also, the second amount of syngas can include additional syngas that was not introduced to the first reaction zone. In some embodiments, the second amount of syngas includes syngas that was generated from methanol in the first reaction zone.

In some embodiments, the first reaction zone is in a first reactor, the second reaction zone is in a second reactor, and an output stream of the first reactor comprises syngas introduced from the first reaction zone into the second reaction zone, further comprising separating from the output stream at least a portion of the methanol produced in the first reaction zone. The first reaction zone and second reaction zone can both be in a single reactor.

In certain embodiments, additional or recycled CO₂ can be introduced into the first reaction zone, wherein at least a portion of the CO₂ is reacted with H₂ present to produce CO₂-derived methanol. The CO₂-derived methanol can be converted, at least in part, to one or more C₂-C₄ alcohols (such as ethanol) in the second reaction zone.

Generally, this invention describes a method of producing an intermediate lower alcohol that is used to produce a higher alcohol. A Cₙ₊ₘ(n+m=2−10) alcohol can be produced by first producing a Cₙ (n=1−5) alcohol, according to the steps of:
(i) introducing syngas into a first reaction zone comprising at least a first catalyst;
(ii) converting a portion of the syngas to a Cₙ alcohol with the first catalyst;
(iii) introducing syngas and the Cₙ alcohol from the first reaction zone into a second reaction zone comprising at least a second catalyst; and
(iv) converting at least a portion of the syngas and the Cₙ alcohol introduced into the second reaction zone with the second catalyst to produce a product stream comprising the Cₙ₋ₘ alcohol.

In some embodiments, intermediate production of a higher alcohol can be used to produce a lower alcohol as a final product. Specifically, methods can include converting at least a portion of the syngas and the Cₙ alcohol introduced into the second reaction zone with the second catalyst to produce a stream comprising an alcohol that is at least one carbon number smaller than the Cₙ alcohol, wherein n is selected from 2 to 5.

### BRIEF DESCRIPTION OF THE FIGURES

FIG. 1 shows a process flow for producing methanol and ethanol from syngas using two reactors in sequence, according to one variation.

FIG. 2 shows a process flow for producing methanol and ethanol from syngas using two reaction zones in sequence in a single reactor, according to one variation.

FIG. 3 shows a process flow for producing methanol and ethanol from syngas using two reactors in sequence, with some or all of the methanol produced in the first reactor diverted from the second reactor, according to one variation.

FIG. 4 shows a process flow for producing methanol and ethanol from syngas using two reactors in sequence according to another variation.

FIG. 5 shows a process flow for producing methanol and ethanol from syngas using two reactors in sequence, with the first reactor producing methanol in high yield for conversion to ethanol in the second reactor, according to one variation.

These and other embodiments, features, and advantages of the present invention will become more apparent to those skilled in the art when taken with reference to the following more detailed description of the invention in conjunction with the accompanying drawings that are first briefly described.

### DETAILED DESCRIPTION OF EMBODIMENTS OF THE INVENTION

Certain embodiments of the present invention will now be further described in more detail, in a manner that enables the claimed invention so that a person of ordinary skill in this art can make and use the present invention.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used herein, “C₁-C₄ alcohols” means one or more alcohols selected from methanol, ethanol, propanol, and butanol, including all known isomers of such compounds. While some embodiments are described in relation to high selectivities to ethanol, the invention can also be practiced in a manner that gives high selectivities to methanol, propanol, and/or butanol, or certain combinations of selectivities to methanol, ethanol, propanol, and butanol. “C₂-C₄ alcohols” means one or more alcohols selected from ethanol, propanol, and butanol, including all known isomers of such compounds.

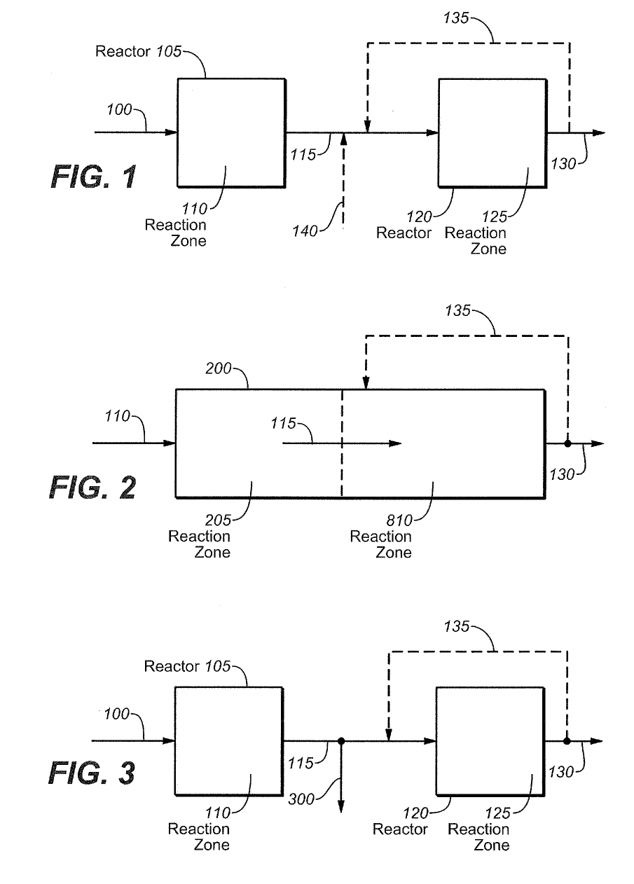

Methods and apparatus for producing C₁-C₄ alcohols from syngas are disclosed herein. In some variations of these methods and apparatus, syngas is catalytically converted to methanol in a first reaction zone, and residual syngas from the first reaction zone is then catalytically converted to ethanol in a second reaction zone. Referring to FIG. 1, for example, in one variation a syngas feedstream 100 is introduced into a first reactor 105 comprising a first reaction zone 110. One or more catalysts in reaction zone 110 convert at least a portion of syngas feedstream 100 to methanol to provide an intermediate product stream 115 comprising at least a portion of the residual (unreacted) syngas from feedstream 100, methanol, and, in some variations, higher alcohols and/or other reaction products.

At least a portion of intermediate product stream 115 is introduced into a second reactor 120 comprising a second reaction zone 125. One or more catalysts in reaction zone 125 convert at least a portion of syngas from intermediate product stream 115 and/or at least a portion of methanol from intermediate product stream 115 to provide a product stream 130 comprising ethanol and, in some variations, methanol, higher alcohols, other reaction products, and/or unreacted syngas from intermediate product stream 115.

Various components of product stream 130 such as, for example, methanol, ethanol, alcohol mixtures (e.g., methanol, ethanol, and/or higher alcohols), water, and unreacted syngas may be separated out and (optionally) purified by the methods described herein or by conventional methods. Such methods may include, for example, distillation and membrane separation processes as well as drying or purifying with molecular sieves.

Syngas feedstream 100 may be produced in any suitable manner known to one of ordinary skill in the art from any suitable feedstock. In some variations, syngas feedstream 100 is filtered, purified, or otherwise conditioned prior to being introduced into reactor 105. For example, carbon dioxide, benzene, toluene, ethyl benzene, xylenes, sulfur compounds, metals, and/or other impurities or potential catalyst poisons may be removed from syngas feedstream 100 by conventional methods known to one of ordinary skill in the art.

In some variations, syngas feedstream 100 comprises H₂ and CO at a H₂/CO ratio having a value between about 0.5 to about 3.0, about 1.0 to about 1.5, or about 1.5 to about 2.0. The H₂/CO ratio in feedstream 100 can, in some variations, affect the yield of methanol and other products in reactor 105. The preferred H₂/CO ratio in such variations may depend on the catalyst or catalysts used in reactor 105 as well as on the operating conditions. Consequently, in some variations, the production and/or subsequent conditioning of syngas feedstream 100 is controlled to produce syngas having a H₂/CO ratio within a range desired to optimize, for example, production of methanol, ethanol, or both methanol and ethanol.

Syngas feedstream 100 may optionally be pressurized and/or heated by compressors and heaters (not shown) prior to entering reactor 105. In some variations, syngas feedstream 100 enters reactor 105 at a temperature of about 300° F. to about 600° F. and at a pressure of about 500 psig to about 2500 psig. In some embodiments, the temperature is between about 300° F. to about 400° F., about 400° F. to about 500° F., or about 500° F. to about 600° F. In some embodiments, the pressure is about 500 psig to about 1000 psig, about 1000 psig to about 2000 psig, or about 2000 psig to about 2500 psig.

Reactor 105 may be any type of catalytic reactor suitable for the conversion of syngas to methanol, alcohol mixtures comprising methanol, higher alcohols, and/or other products. Reactor 105 may, for example, be any suitable fixed-bed reactor. In some variations, reactor 105 comprises tubes filled with one or more catalysts. Syngas passing through the tubes undergoes catalyzed reactions to form methanol and, in some variations, higher alcohols or other products. In some embodiments, catalysis occurs within pellets or in a homogeneous phase.

Reactor 105 may operate, for example, at temperatures of about 400° F. to about 700° F. and at pressures of about 500 psig to about 2500 psig. In some embodiments, the temperature is between about 400° F. to about 500° F., about 500° F. to about 600° F., or about 600° F. to about 700° F. In some embodiments, the pressure is about 500 psig to about 1000 psig, about 1000 psig to about 2000 psig, or about 2000 psig to about 2500 psig.

In some embodiments, conditions effective for producing alcohols from syngas include average reactor residence times from about 0.1-10 seconds, preferably about 0.5-2 seconds. “Average reactor residence time” is the mean of the residence-time distribution of the reactor contents under actual operating conditions. Catalyst contact times can also be calculated by a skilled artisan and these times will typically also be in the range of 0.1-10 seconds, although it will be appreciated that it is certainly possible to operate at shorter or longer times.

The reactor for converting syngas into alcohols can be engineered and operated in a wide variety of ways. The reactor operation can be continuous, semicontinuous, or batch. Operation that is substantially continuous and at steady state is preferable. The flow pattern can be substantially plug flow, substantially well-mixed, or a flow pattern between these extremes. The flow direction can be vertical-upflow, vertical-downflow, or horizontal. A vertical configuration can be preferable.

The “reactor” can actually be a series or network of several reactors in various arrangements. For example, in some variations, the reactor comprises a large number of tubes filled with one or more catalysts.

Any suitable catalyst or combination of catalysts may be used in reactor 105 to catalyze reactions converting syngas to methanol and, optionally, to higher alcohols and/or other products. Suitable catalysts may include, but are not limited to, one or more of ZnO/Cr₂O₃, Cu/ZnO, Cu/ZnO/Al₂O₃, Cu/ZnO/Cr₂O₃, Cu/ThO₂, Co/Mo/S, Co/S, Mo/S, Ni/S, Ni/Mo/S, Ni/Co/Mo/S, Rh, Ti, Fe, Ir, and any of the foregoing in combination with Mn and/or V. The addition of basic promoters (e.g. K, Li, Na, Rb, Cs, and Fr) increases the activity and selectivity of some of these catalysts for alcohols. Basic promoters include alkaline-earth and rare-earth metals. Non-metallic bases can also serve as effective promoters, in some embodiments.

The catalyst phase can be a packed bed or a fluidized bed. The catalyst particles can be sized and configured such that the chemistry is, in some embodiments, mass-transfer-limited or kinetically limited. The catalyst can take the form of a powder, pellets, granules, beads, extrudates, and so on. When a catalyst support is optionally employed, the support may assume any physical form such as pellets, spheres, monolithic channels, etc. The supports may be coprecipitated with active metal species; or the support may be treated with the catalytic metal species and then used as is or formed into the aforementioned shapes; or the support may be formed into the aforementioned shapes and then treated with the catalytic species.

In some variations, up to about 50% of CO in syngas feedstream 100 is converted to methanol in reaction zone 110. Intermediate product stream 115 output from reactor 105 may comprise, in some variations, about 5% to about 50% methanol, about 5% to about 50% ethanol, about 5% to about 25% CO, about 5% to about 25% H₂, and about 2% to about 35% CO₂, as well as other gases. In some embodiments, intermediate product stream 115 also comprises one or more higher alcohols, such as ethanol, propanol, or butanol.

The H₂/CO ratio in intermediate product stream 115 can, in some variations, affect the yield of ethanol and other products in reactor 120. The preferred H₂/CO ratio in such variations may depend on the catalyst or catalysts used in reactor 120 as well as on the operating conditions. The H₂/CO ratio in intermediate product stream 115 can differ from that of feedstream 100 as a result of reactions occurring in reactor 105. In some variations, the H₂/CO ratio of intermediate product stream 115 provides a higher ethanol yield in reactor 120 than would the H₂/CO ratio of feedstream 100. In such variations, operation of reactor 105 to produce methanol, for example, improves the H₂/CO ratio of the syngas fed to reactor 120 from the standpoint of ethanol yield in reactor 120.

In one example, feedstream 100 comprises syngas with an H₂/CO ratio of about 1.5 to about 2, and the preferred H₂/CO ratio for production of ethanol in reactor 120 is about 1. Operation of reactor 105 to produce methanol in this example depletes H₂ in the syngas to decreases the H₂/CO ratio in intermediate product stream 115 to a value closer to 1 and thus improves the ethanol yield in reactor 120. In certain embodiments, the catalyst in reactor 105 is a Cu/ZnO/alumina catalyst.

Reactor 120 may be any type of catalytic reactor suitable for the conversion of syngas, methanol, and/or syngas plus methanol to ethanol and, optionally, to higher alcohols and/or other products. Reactor 120 may be any suitable fixed-bed reactor, for example. In some variations, reactor 120 comprises tubes filled with one or more catalysts. Syngas and/or methanol passing through the tubes undergoes surface catalyzed reactions to form ethanol and, in some variations, higher alcohols and/or other products.

While not intending to be bound by any particular theory, it is presently believed that the methanol may be converted to syngas and thence to ethanol, the methanol may be converted directly to ethanol via a homologation reaction, and/or the methanol may be converted to ethanol by other mechanisms.

Reactor 120 may operate, for example, at temperatures of about 500° F. to about 800° F. and at pressures of about 500 psig to about 2500 psig. In some embodiments, the temperature is between about 500° F. to about 600° F., about 600° F. to about 700° F., or about 700° F. to about 800° F. In some embodiments, the pressure is about 500 psig to about 1000 psig, about 1000 psig to about 2000 psig, or about 2000 psig to about 2500 psig.

Any suitable catalyst or combination of catalysts may be used in reactor 120 to catalyze reactions converting syngas, methanol, and/or syngas +methanol to ethanol and, optionally, to higher alcohols and/or other products. Suitable catalysts may include, but are not limited to, alkali/ZnO/Cr₂O₃, Cu/ZnO, Cu/ZnO/Al₂O₃, CuO/CoO, CuO/CoO/Al₂O₃, Mo/S, Co/Mo/S, Ni/S, Ni/Mo/S, Ni/Co/Mo/S, Rh/Ti/SiO₂, Rh/Mn/SiO₂, Rh/Ti/Fe/Ir/SiO₂, Rh/Mn/MCM-41, Cu, Zn, Rh, Ti, Fe, Ir, and mixtures thereof. The addition of basic promoters (e.g. K, Li, Na, Rb, Cs, and Fr) increases the activity and selectivity of some of these catalysts for ethanol or other C₂₊ alcohols. Basic promoters include alkaline-earth and rare-earth metals. Non-metallic bases can also serve as effective promoters, in some embodiments.

In some embodiments, catalysts for reactor 120 can include one or more of ZnO/Cr₂O₃, Cu/ZnO, Cu/ZnO/Al₂O₃, CuO/CoO, CuO/CoO/Al₂O₃, Co/S, Mo/S, Co/Mo/S, Rh/Ti/SiO₂, Rh/Mn/SiO₂, Rh/Ti/Fe/Ir/SiO₂, Rh/Mn/MCM-41, Ni/S, Ni/Mo/S, Ni/Co/Mo/S, and any of the foregoing in combination with Mn and/or V. Again, any of these catalysts can (but do not necessarily) include one or more basic promoters.

The composition of catalysts in reactors 105 and 120, or reaction zones 110 and 125, can be similar or even the same. Reference to a “first catalyst” and “second catalyst” in conjunction with reaction zones is a reference to different physical materials, not necessarily a reference to different catalyst compositions. In some embodiments, a certain type of catalyst is loaded into both reaction zones but, over time, the nominal composition of these catalysts could diverge somewhat due to different exposure conditions.

Product stream 130 output from reactor 120 may comprise, in some variations, about 0% to about 50% methanol, about 10% to about 90% ethanol, about 0% to about 25% CO, about 0% to about 25% H₂, and about 5% to about 25% CO₂, as well as other gases. In some embodiments, product stream 130 also comprises one or more higher alcohols, such as propanol or butanol.

Referring again to FIG. 1, in some variations unreacted syngas in product stream 130 is separated from product stream 130 to form feedstream 135 and recycled through reactor 120 to further increase, for example, the yield of ethanol and/or other desired products. Alternatively, or in addition, in some variations unreacted syngas in product stream 130 is recycled through reactor 105 by adding it to syngas feedstream 100. The latter approach may be unsuitable, however, if the unreacted syngas in product stream 130 is contaminated, for example, with sulfur, sulfur compounds, metals, or other materials that can poison methanol catalysts in reactor 105.

Also, in some variations a methanol feedstream 140 is added to intermediate product stream 115 or otherwise introduced to reactor 120 to further increase, for example, the yield of ethanol and/or other desired products. For example, methanol in product stream 130 may be separated (not shown) from product stream 130 to form feedstream 140 and then recycled through reactor 120. Methanol from other sources may be introduced, as well or instead, into reactor 120.

In some variations, one or more catalysts in reactor 105, one or more catalysts in reactor 120, or one or more catalysts in both reactor 105 and reactor 120 catalyze the conversion of CO₂ to methanol. Production of methanol in reactor 105, reactor 120, or in both reactors may be thereby enhanced by consumption of CO₂ present in syngas feedstream 100. Consequently, in some variations, CO₂ is added to syngas feedstream 100 or the production and/or subsequent conditioning of syngas feedstream 100 is controlled to produce syngas having a desirable amount of CO₂. Suitable catalysts for converting CO₂ to methanol may include, in some variations, one or more of those listed above for use in reactor 105 and reactor 120. Enhanced production of methanol by consumption of CO₂ may result, in some variations, in enhanced production of ethanol by conversion of the methanol to ethanol and/or by a resulting favorable adjustment of the H₂/CO ratio in the syngas stream introduced to reactor 120.

Referring now to FIG. 2, some alternative variations differ from those described above primarily by use of a single reactor 200 comprising a first reaction zone 205 and a second reaction zone 810 rather than two reactors. Syngas feedstream 100 is introduced into first reaction zone 205, wherein one or more catalysts convert at least a portion of syngas feedstream 100 to methanol to provide intermediate product stream 115 comprising at least a portion of the unreacted syngas from feedstream 100, methanol, and, in some variations, higher alcohols and/or other reaction products. At least a portion of intermediate product stream 115 is introduced into second reaction zone 810, where one or more catalysts convert at least a portion of syngas from intermediate product stream 115 and/or at least a portion of methanol from intermediate product stream 115 to provide product stream 130 comprising ethanol and, in some variations, methanol, higher alcohols, other reaction products, and/or unreacted syngas from intermediate product stream 115.

Reactor 200 may be any type of suitable catalytic reactor comprising two or more reaction zones. Operation of reactor 200 may be similar to the operation of reactors 105 and 120 described above. In particular, in some variations, the catalysts used in reactions zones 205 and 810 and the operating conditions for the reaction zones are the same as or similar to those for, respectively, reaction zones 110 and 120 described above. The compositions of intermediate product stream 115 and product stream 130 may, in some variations, be the same as or similar to those for the variations described above with respect to FIG. 1. Syngas in product stream 130 may be recycled through reaction zone 810 or added to feedstream 100. CO₂ may be added to syngas feedstream 100 or the production and/or subsequent conditioning of syngas feedstream 100 may be controlled to produce syngas having a desirable amount of CO₂ for enhanced methanol production. A methanol feedstream (not shown) may be introduced to reaction zone 810 to further increase, for example, the yield of ethanol and/or other desired products. This methanol feedstream may be separated from product stream 130, for example.

Similarly to the two-reactor variations, in some of the single-reactor variations the H₂/CO ratio in intermediate product stream 115 can affect the yield of ethanol and other products in reaction zone 810. In some variations, the H₂/CO ratio of intermediate product stream 115 differs from that of feedstream 100 and provides a higher ethanol yield in reaction zone 810 than would the H₂/CO ratio of feedstream 100. In such variations, production of methanol in reaction zone 205, for example, improves the H₂/CO ratio of the syngas fed to reaction zone 810 from the standpoint of ethanol yield in reactor 120.

Referring now to FIG. 3, some alternative variations differ from those described with respect to FIG. 1 in that at least a portion (some or substantially all) of the methanol in intermediate product stream 115 is diverted into a methanol product stream 300 prior to the introduction of product stream 115 into reactor 120. Methanol in product stream 300 can be separated and purified by conventional methods. Similarly as above, in some of these variations, the H₂/CO ratio of intermediate product stream 115 differs from that of feedstream 100 and provides a higher ethanol yield in reactor 120 than would the H₂/CO ratio of feedstream 100. Hence, the production of methanol in reactor 105 may advantageously enhance ethanol production in reactor 120 in some of these variations.

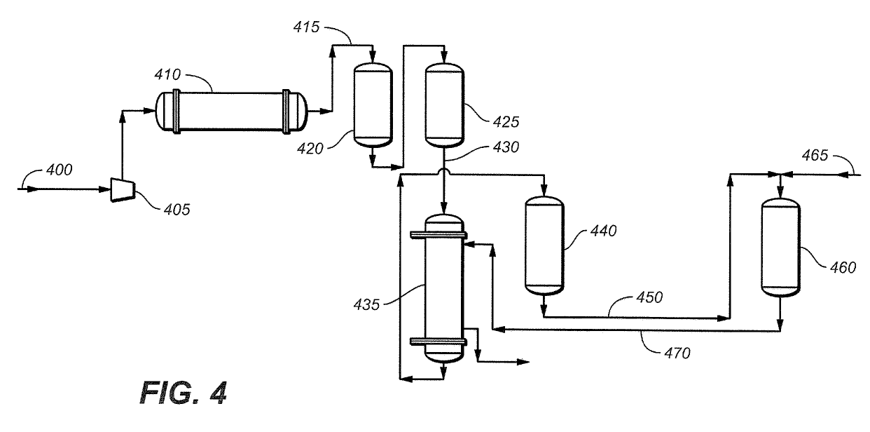

In some variations methanol is produced at high yield in a first reactor and subsequently converted to ethanol in a second reactor. One example is described with reference to FIG. 4 described in more detail below.

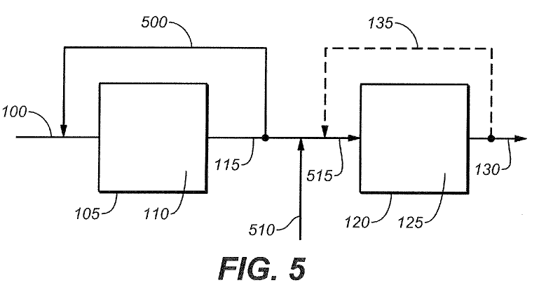

Referring to FIG. 5, for example, in some variations a syngas feedstream 100 is catalytically converted to methanol in a first reactor 105 at a yield (mole conversion of CO to methanol) of, for example, at least about 50%, preferably at least about 75% or even higher. Such high methanol yields may be facilitated, for example, by separating out some or substantially all of the non-methanol components in intermediate product stream 115 as a stream 500 that is recycled through reactor 105.

An unrecycled portion of intermediate product stream 115, rich in methanol, is (optionally) mixed with another syngas feedstream 510 to provide feedstream 515 which is introduced into reactor 120. At least a portion of the methanol and (optionally) syngas introduced into reactor 120 is catalytically converted to provide a product stream 130 comprising ethanol and, in some variations, methanol, higher alcohol, other reaction products, and/or unreacted syngas from feedstream 515. In some variations, unreacted syngas in product stream 130 is recycled through reactor 120 as feedstream 135 and/or recycled through reactor 105. Various components of product stream 130 may be separated out and/or purified as described above.

In some variations, the ratio of methanol to CO in feedstream 100 may be adjusted, for example, to optimize the yield of ethanol in reactor 120. In some embodiments, the molar ratio of methanol/CO in reactor 120 is between about 0.5 to about 2.0. In particular embodiments, the ratio of methanol/CO in reactor 120 is about 1.0.

Any suitable catalyst or combination of catalysts may be used in reactor 105. Suitable catalysts for reactor 105 may include, but are not limited to, the methanol catalysts listed above. Similarly, any suitable catalyst or combination of catalysts may be used in reactor 120. Suitable catalysts for reactor 120 may include, but are not limited to, the ethanol catalysts listed above. The composition of catalysts in reactors 105 and 120 can be similar or even substantially the same.

In variations of any of the methods described herein that use a first reaction zone and a second reaction zone, the initial syngas stream can be introduced into both the first reaction zone and the second reaction zone. In some embodiments, the syngas is from an external source. In some embodiments, the syngas is from any of the methods described herein (such as residual syngas from a first reaction zone or a second reaction zone).

In some embodiments of any of the methods described herein, syngas from any source is added to the first reaction zone and/or the second reaction zone. In some embodiments of any of the methods described herein, methanol from any source is added to the second reaction zone.

Certain embodiments employ a plurality of physical reactors in one or both of the reaction zones. For example, the first zone could consist of two reactors, followed by a single reactor as the second zone. Or, in another example, the first zone could be one reactor followed by two reactors in the second zone. In general, any “zone” or “reaction zone” can contain a fraction of one, two, three, or more physical reactors.

In some embodiments of any of the methods described herein, reaction conditions (such as the temperature and pressure) used for the conversion of syngas to methanol, the conversion of syngas and/or methanol to ethanol, or the homologation of methanol to ethanol are the same as those described in any of U.S. Pat. Nos. 4,371,724; 4,424,384; 4,374,285; 4,409,405; 4,277,634; 4,253,987; 4,233,466; and 4,171,461; all of which are incorporated by reference herein in their entirety.

FIG. 4 shows an example of a process in which syngas is catalytically converted to methanol in a first reactor, and methanol and residual syngas from the first reactor are converted to ethanol in a second reactor. Referring now to FIG. 4, a single two-stage intercooled reciprocating compressor 405 compresses syngas feedstream 400 to about 1500 psig and feeds it at a temperature of about 135° F. to syngas preheater 410. Preheater 410 is a shell and tube heat exchanger that uses steam as an enthalpy source.

In this example associated with FIG. 4, heated syngas 415 from preheater 410 is sent to a set of reactor guard beds 420, 425. Guard beds 420, 425 are configured in a permanent lead-lag arrangement but are piped such that either bed can be bypassed. The piping arrangement allows one bed to be in service while the other is being regenerated or activated. Regeneration is initiated by a mixed hydrogen and nitrogen line (not shown). Guard beds 415, 420 remove, for example, sulfurs and metals that may poison the methanol catalysts. In some embodiments, one or more catalyst poisons are removed by adsorption over copper, copper chromite, nickel, cobalt, or molybdenum. These and other metals can be supported on high-surface-area refractory inorganic oxide materials such as alumina, silica, silica/alumina, clays, or kieselguhr. One exemplary material is copper on alumina. Exit gases 430 from guard beds 420, 425 are sent to an alcohol reactor cross exchanger 435 at about 350° F. and are heated to about 480° F. during heat exchange with crude alcohol exit gases 470 from second alcohol reactor 460.

With continuing reference to FIG. 4, syngas at about 1500 psig and about 480° F. enters a first alcohol synthesis reactor 440, where at least a portion of the syngas undergoes a catalyzed reaction in supported-catalyst tubular reactors within the reactor vessel. In some variations, the catalyst in reactor 440 is a Cu/ZnO/alumina catalyst. Methanol is expected to be formed via the reaction CO+2 H₂→CH₃OH. As noted earlier in this detailed description, in some variations methanol may be formed by the hydrogenation of CO₂ as well.

Product gases 450 leave alcohol synthesis reactor 440 at a temperature of about 500° F. and enter alcohol synthesis reactor 460. In addition, a methanol stream 465 (e.g., a methanol recycle stream separated from crude alcohol stream 470) is mixed with the product gases 450 from reactor 440 and also introduced to reactor 460. Reactions occurring in reactor 460 can include ethanol formation.

Crude alcohol stream 470 exits reactor 460 at a temperature of about 650° F. and is cooled by heat exchange in alcohol reactor cross exchanger 435 to a temperature of about 530° F. Subsequent heat recovery and other cooling steps (not shown) cool crude alcohol stream 470 to about 100° F. Ethanol, methanol, residual syngas, and other components of crude alcohol stream 470 may be separated and (optionally) purified by using the methods described herein or using conventional methods (not shown). Syngas recovered from stream 470 may, for example, be recycled through the reactors by mixing it with syngas feedstream 400.

Some variations may employ microwave, radio frequency, laser, and/or UV energy in addition to or instead of conventional process heat (e.g., steam, heat from burners, waste heat, etc.) to facilitate the production of ethanol. For example, microwave, radio frequency, laser, and/or UV energy may be used in some variations to convert CO₂ in syngas to CO and 02 for more efficient catalytic conversion to methanol and/or ethanol. In some embodiments, a conventional method for converting CO₂ in syngas to CO (e.g., treating syngas with a catalyst that promotes the conversion of CO₂ to CO) is used for more efficient catalytic conversion to methanol and/or ethanol. In some embodiments, both a catalyst and irradiation (such as irradiation with microwave, radio frequency, laser, and/or UV energy) are used to convert CO₂ to CO. In particular embodiments, CO₂ is removed from the syngas and irradiation (such as irradiation with microwave, radio frequency, laser, and/or UV energy) and/or a catalyst (such as a thermal catalyst) is used to generate 02 from CO. The 02 is removed and the CO is added to the first and/or second reactor zone. In some embodiments, the irradiation allows a lower temperature and/or pressure to be used for conversion of CO₂ to CO than the standard temperatures and pressures used for conversion of CO₂ to CO without irradiation. CO₂ in syngas stream 100 may be optionally converted in this manner in some variations.

As another example, microwave, radio frequency, laser, and/or UV energy may be used to accelerate the catalytic conversion of syngas to methanol and/or ethanol, and/or to accelerate the catalytic conversion of syngas and/or methanol to ethanol in variations of the processes described above for conversion of syngas to ethanol. More generally, in some variations, microwave, radio frequency, laser, and/or UV energy may be used to accelerate the catalytic conversion of syngas of any origin to methanol and/or ethanol, and/or to accelerate the catalytic conversion of syngas and/or methanol of any origin to ethanol.

In some embodiments, microwave, radio frequency, laser, and/or UV energy is used to irradiate syngas and/or the first catalyst in the first reaction zone to enhance the conversion of syngas to methanol. In some embodiments, the irradiation increases molecular vibrations, increases the energy density, or otherwise activates the syngas and/or first catalyst. Such use of microwave, radio frequency, laser, and/or UV energy in a syngas-to-methanol reactor, for example, may allow the reactor to be operated at lower temperatures and pressures than otherwise.

In some variations, microwave, radio frequency, laser, and/or UV energy is used to irradiate the syngas, methanol, and/or the second catalyst in the second reaction zone. In some embodiments, the irradiation increases molecular vibrations, increases the energy density, or otherwise activates the syngas, methanol, and/or second catalyst. Enhancement of catalytic conversion of methanol to ethanol may occur, for example, by preferential absorption of the microwave, radio frequency, laser, and/or UV energy by the methanol allowing high energy densities to be achieved in the methanol reactants. For example, microwaves heat methanol at a faster rate than ethanol, thereby favoring the conversion of methanol to ethanol. Such use of microwave, radio frequency, laser, and/or UV energy in a methanol to ethanol reactor, for example, may allow the reactor to be operated at lower temperatures and pressures than otherwise.

In some embodiments, methods involve introducing syngas into a reaction zone (e.g., a reactor) comprising at least one catalyst, and irradiating the syngas and/or the catalyst in the reaction zone with energy (e.g., microwave, radio frequency, laser, and/or UV energy). At least a portion of the syngas can be converted to ethanol. The method may also produce methanol or other alcohols. Suitable catalysts may include, but are not limited to, any of the catalysts described herein. In some embodiments, the catalyst is a conventional catalyst for the conversion of syngas to ethanol in one reaction zone or one reactor. In some embodiments, the catalyst favors the formation of ethanol over methanol in the absence of irradiation, and the irradiation enhances the selectivity for the formation of ethanol. For example, the irradiation may heat methanol at a faster rate than ethanol, thereby favoring the conversion of methanol to ethanol. In some embodiments, the catalyst favors the formation of methanol over ethanol in the absence of irradiation, and the irradiation causes the catalyst to produce a lower ratio of methanol to ethanol than in the absence of irradiation. For example, irradiation may cause the catalyst to now produce more ethanol than methanol.

In other embodiments, methods involve introducing syngas and/or methanol into a reaction zone comprising at least one catalyst, and irradiating the syngas, methanol, and/or the catalyst in the reaction zone with energy (e.g., microwave, radio frequency, laser, and/or UV energy). At least a portion of the syngas and/or methanol is converted to ethanol. The method may also produce other alcohols. In particular embodiments, both syngas and methanol are introduced in to the reaction zone. In some embodiments, either syngas or methanol is introduced in to the reaction zone. In some embodiments, methanol is produced using any of the methods described herein or obtained from any other source, and the methanol without syngas is introduced in to the reactor zone. Suitable catalysts may include, but are not limited to, any of the catalysts described herein.

In some embodiments, ethanol is purified from the product stream 130 or crude alcohol stream 470 by first drying the product stream 130 or crude alcohol stream 470 to produce an intermediate product and then distilling the intermediate product to produce a purified ethanol product. In some embodiments, the product stream 130 or crude alcohol stream 470 comprises ethanol, methanol, propanol, butanol, and water. In some embodiments, product stream 130 or crude alcohol stream 470 includes one or more of the following alcohols: 1-propanol, 2-propanol, 1-butanol, 2-butanol, t-butanol, pentanols, hexanols, heptanols, and octanols, and/or higher alcohols. In some embodiments, product stream 130 or crude alcohol stream 470 includes one or more aldehydes, ketones, and/or organic acids (such as formaldehyde, acetaldehyde, acetic acid, and the like).

In particular embodiments, the amount of the ethanol is between about 25% to about 95% of the product stream 130 or crude alcohol stream 470 by weight, such as between about 30% to about 50% or between about 50% to about 90% by weight. In particular embodiments, the amount of the methanol is between about 1% to about 50% of the product stream 130 or crude alcohol stream 470 by weight, such as between about 5% to about 25% or between about 25% to about 55% by weight. In particular embodiments, the amount of the water is between about 1% to about 50% of the product stream 130 or crude alcohol stream 470 by weight, such as between about 1% to about 10%, or about 10% to about 20%. In particular embodiments, the amount of the propanol is between about 0.5% to about 10% of the product stream 130 or crude alcohol stream 470 by weight, such as between about 1% to about 2% or between about 2% to about 8% by weight. In particular embodiments, the butanol is between about 0.2% to about 5% of the product stream 130 or crude alcohol stream 470 by weight, such as between about 0.5% to about 2% or between about 2% to about 5% by weight.

In particular embodiments, the combined amount of ketones and aldehydes is between about 0.1% to about 10% of the product stream 130 or crude alcohol stream 470 by weight, such as between about 0.5% to about 2%. In particular embodiments, the combined amount of organic acids is between about 0.1% to about 10% of the product stream 130 or crude alcohol stream 470 by weight, such as between about 0.5% to about 2%. In particular embodiments, the combined amount of C₅ and higher alcohols is between about 0.1% to about 5% of the product stream 130 or crude alcohol stream 470 by weight, such as between about 0.5% to about 2%.

In particular embodiments, drying is performed prior to distillation, rather than after distillation. A drying step can reduce the amount of water in the product stream 130 or crude alcohol stream 470 by at least 75%, preferably at least 90%, more preferably at least 95%, and most preferably at least about 99%. In particular embodiments, the amount of the water is less than or equal to about 1% or less of the intermediate product by weight. Drying can also be referred to as “dehydration” which herein means removal of water from solution, not removal of water at the molecular level (such as during olefin formation).

In some embodiments, the drying step involves passing the product stream 130 or crude alcohol stream 470 through a membrane, such as zeolite membrane, or through one or more molecular sieves to produce an intermediate product. In some embodiments, the molecular sieve has an effective pore size of less than about 5 Angstroms. In certain embodiments, the molecular sieve has an effective pore size of about 3 Angstroms.

In other embodiments, the drying step involves passing the product stream 130 or crude alcohol stream 470 through a desiccant. A large variety of desiccants are known. For example, desiccants can be selected from SiO₂, CaO, CaCO₃, CaCl₂, CuSO₄, or CaSO₄.

Conventional distillation methods, well-known in the art, can be used to distill the intermediate product. Any number of distillation columns may be employed, depending on the desired overall separation. In some embodiments, ethanol is between about 95% to about 99.9% of the purified product by weight. The purified ethanol product can be made to meet the ASTM D4806-07a specification for fuel ethanol, or some other fuel-grade specification as will be appreciated.

The purified ethanol product can be used to power an internal combustion engine to power a transportation vehicle. In some embodiments, the purified ethanol product can be combined (blended) with at least one other hydrocarbon, or multiple hydrocarbons such as gasoline, to create a liquid-fuel blend.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are incorporated herein by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A method of producing one or more C₂-C₄ alcohols, said method comprising:
(i) introducing syngas into a first reaction zone comprising at least a first catalyst;
(ii) converting a portion of said syngas to methanol with said first catalyst;
(iii) introducing syngas and methanol from said first reaction zone into a second reaction zone comprising at least a second catalyst; and
(iv) converting at least a portion of said syngas and methanol introduced into said second reaction zone with said second catalyst to produce a product stream comprising one or more C₂-C₄ alcohols.

2. The method of claim 1, wherein said C₂-C₄ alcohols comprise ethanol.

3. The method of claim 1, wherein said C₂-C₄ alcohols comprise 1-propanol.

4. The method of claim 1, wherein said C₂-C₄ alcohols comprise 1-butanol.

5. The method of claim 1, wherein said syngas introduced into said first reaction zone has an initial H₂/CO ratio, conversion of syngas to methanol in said first reaction zone causes said syngas introduced into said second reaction zone to have a second H₂/CO ratio, and said second H₂/CO ratio provides an increased yield to one or more C₂-C₄ alcohols in said second reaction zone compared to that which would be provided by said initial H₂/CO ratio.

6. The method of claim 5, wherein said second H₂/CO ratio is lower than said initial H₂/CO ratio.

7. The method of claim 1, further comprising introducing additional methanol into said second reaction zone and converting at least a portion of said additional methanol introduced into said second reaction zone to one or more C₂-C₄ alcohols with said second catalyst.

8. The method of claim 7, wherein at least a portion of said additional methanol introduced into said second reaction zone was previously recovered from said product stream.

9. The method of claim 1, further comprising introducing additional syngas that is not unreacted syngas from said first reaction zone, into said second reaction zone and converting at least a portion of said additional syngas introduced into said second reaction zone to one or more C₂-C₄ alcohols with said second catalyst.

10. The method of claim 1, further comprising recovering syngas from said product stream and recycling the recovered syngas through at least one of said reaction zones.

11. The method of claim 1, wherein said first catalyst comprises a material selected from the group consisting of ZnO/Cr₂O₃, Cu/ZnO, Cu/ZnO/Al₂O₃, Cu/ZnO/Cr₂O₃, Cu/ThO₂, Co/S, Mo/S, Co/Mo/S, Ni/S, Ni/Mo/S, Ni/Co/Mo/S, and any of the foregoing in combination with Mn and/or V, and wherein said first catalyst optionally includes a basic promoter.

12. The method of claim 1, wherein said second catalyst comprises a material selected from the group consisting of ZnO/Cr₂O₃, Cu/ZnO, Cu/ZnO/Al₂O₃, CuO/CoO, CuO/CoO/Al₂O₃, Co/S, Mo/S, Co/Mo/S, Rh/Ti/SiO₂, Rh/Mn/SiO₂, Rh/Ti/Fe/Ir/SiO₂, Rh/Mn/MCM-41, Ni/S, Ni/Mo/S, Ni/Co/Mo/S, and any of the foregoing in combination with Mn and/or V, and wherein said second catalyst optionally includes a basic promoter.

13. The method of claim 1, wherein said first catalyst and said second catalyst have substantially the same initial composition.

14. The method of claim 1, wherein said first reaction zone and second reaction zone are both in a single reactor.

15. A method of producing one or more C₂-C₄ alcohols, said method comprising:
(i) introducing a first amount of syngas into a first reaction zone comprising at least a first catalyst;
(ii) converting at least a portion of said first amount of syngas to methanol with said first catalyst;
(iii) introducing said methanol to a second reaction zone comprising at least a second catalyst;
(iv) introducing a second amount of syngas to said second reaction zone; and
(v) reacting at least a portion of said methanol introduced into said second reaction zone with at least a portion of said second amount of syngas with said second catalyst to produce a product stream comprising one or more C₂-C₄ alcohols.

16. The method of claim 15, wherein said second amount of syngas includes syngas that did not react in said first reaction zone.

17. The method of claim 15, wherein said second amount of syngas includes syngas that was separated and recycled from said product stream.

18. The method of claim 15, wherein said second amount of syngas includes additional syngas that was not introduced to said first reaction zone.

19. The method of claim 15, wherein said second amount of syngas includes syngas that was generated from methanol in said first reaction zone.

20. The method of claim 15, wherein said first catalyst comprises a material selected from the group consisting of ZnO/Cr₂O₃, Cu/ZnO, Cu/ZnO/Al₂O₃, Cu/ZnO/Cr₂O₃, Cu/ThO₂, Co/S, Mo/S, Co/Mo/S, Ni/S, Ni/Mo/S, Ni/Co/Mo/S, and any of the foregoing in combination with Mn and/or V, and wherein said first catalyst optionally includes a basic promoter.

21. The method of claim 15, wherein said second catalyst comprises a material selected from the group consisting of ZnO/Cr₂O₃, Cu/ZnO, Cu/ZnO/Al₂O₃, CuO/CoO, CuO/CoO/Al₂O₃, Co/S, Mo/S, Co/Mo/S, Rh/Ti/SiO₂, Rh/Mn/SiO₂, Rh/Ti/Fe/Ir/SiO₂, Rh/Mn/MCM-41, Ni/S, Ni/Mo/S, Ni/Co/Mo/S, and any of the foregoing in combination with Mn and/or V, and wherein said second catalyst optionally includes a basic promoter.

22. The method of claim 15, wherein said first reaction zone is in a first reactor, said second reaction zone is in a second reactor, and an output stream of said first reactor comprises syngas introduced from said first reaction zone into said second reaction zone, further comprising separating from said output stream at least a portion of said methanol produced in said first reaction zone.

23. The method of claim 15, wherein said first reaction zone and second reaction zone are both in a single reactor.

24. The method of claim 15, wherein said first catalyst and said second catalyst have substantially the same initial composition.

25. The method of claim 15, wherein said one or more C₂-C₄ alcohols is selected from the group consisting of ethanol, 1-propanol, and 1-butanol.

26. The method of claim 1 or 15, further comprising introducing additional or recycled CO₂ into said first reaction zone, wherein at least a portion of said CO₂ is reacted with H₂ present to produce CO₂-derived methanol.

27. The method of claim 26, wherein said CO₂-derived methanol is converted, at least in part, to one or more C₂-C₄ alcohols in said second reaction zone.

28. The method of claim 26, wherein said CO₂-derived methanol is converted, at least in part, to ethanol in said second reaction zone.

29. A method of producing a Cₙ₊ₘ alcohol, said method comprising:
(i) introducing syngas into a first reaction zone comprising at least a first catalyst;
(ii) converting a portion of said syngas to a Cₙ alcohol with said first catalyst;
(iii) introducing syngas and said Cₙ alcohol from said first reaction zone into a second reaction zone comprising at least a second catalyst; and
(iv) converting at least a portion of said syngas and said Cₙ alcohol introduced into said second reaction zone with said second catalyst to produce a product stream comprising said Cₙ m alcohol,
wherein n is selected from 1 to 5, and
wherein n+m is selected from 2 to 10.

30. The method of claim 29, further comprising converting at least a portion of said syngas and said Cₙ alcohol introduced into said second reaction zone with said second catalyst to produce a product stream comprising an alcohol that is at least one carbon number smaller than said Cₙ alcohol, wherein n is selected from 2 to 5.
